(19) United States
(12) Patent Application Publication
(10) Pub. No.: US 2018/0303937 A1
(43) Pub. Date: Oct. 25, 2018

(54) A COMPOSITION FOR CONTROLLED RELEASE OF A BIOMOLECULE, METHOD OF PREPARATION AND USES THEREOF

(71) Applicant: CENTRO DE NEUROCIENCIAS E BIOLOGIA CELULAR, Coimbra (PT)

(72) Inventors: Lino DA SILVA FERREIRA, COIMBRA (PT); Miguel Maria DA FONSECA MIRANDA FERREIRA LINO, SALVATERRA DE MAGOS (PT)

(21) Appl. No.: 15/768,757

(22) PCT Filed: Oct. 17, 2016
(86) PCT No.: PCT/IB2016/056229

§ 371 Date: Apr. 16, 2018

(30) Foreign Application Priority Data
Oct. 15, 2015 (PT) 108892

(51) Int. Cl. A61K 41/00; A61K 31/7105; A61K 47/69

(52) U.S. Cl. CPC A61K 41/0042; A61K 31/7105; B82Y 5/00; A61K 47/6923; A61K 47/6929

(57) ABSTRACT

The present disclosure relates to the modulation of cell activity by sequential release of several biomolecule from a given nanocarrier using a given wavelength. The present disclosure describes a composition for controlled release of biomolecules comprising: a gold nanoparticle; at least two different biomolecules; at least two different oligonucleotides having different melting points, binding said biomolecules to the surface of the gold nanoparticle; wherein the binding is obtainable via hybridization of complementary oligonucleotides; wherein each of the oligonucleotides is a photo-active such that the respective biomolecule is releasable by photo-activation. The composition of the present disclosure may be use in medicine, namely in the treatment of cancer, aging diseases, and accelerated aging syndromes, infectious diseases such as AIDS, epigenetic diseases such as polycystic ovary syndrome, or cardiac diseases, such as cardiac regeneration.

### TECHNICAL DOMAIN

The present disclosure relates to the modulation of cell activity by sequential release of several biomolecule, namely peptide, protein or microRNA, from a given nanocarrier using a given wavelength.

### TECHNICAL BACKGROUND

Intracellular delivery of proteins is useful for the manipulation of cellular processes and cell reprogramming (Leader, B., Baca, Q. J. & Golan, D. E. Protein therapeutics: a summary and pharmacological classification. Nat Rev Drug Discov 7, 21-39 (2008)). However, protein transduction has been hindered by the poor membrane permeability of most of the proteins. In the past decade, different nanoformulations have been developed for the delivery of proteins to cells (Kam, N. W. S. & Dai, H. Carbon Nanotubes as Intracellular Protein Transporters: Generality and Biological Functionality. Journal of the American Chemical Society 127, 6021-6026 (2005); Liu, Y. et al. Delivery of Intact Transcription Factor by Using Self-Assembled Supramolecular Nanoparticles. Angewandte Chemie International Edition 50, 3058-3062 (2011)).

However most of these strategies are based on the passive diffusion of the protein from the nanocarrier or on the enzymatic degradation of the nanoformulation (Zhu, S., Nih, L., Carmichael, S. T., Lu, Y. & Segura, T. Enzyme-Responsive Delivery of Multiple Proteins with Spatiotemporal Control. Advanced Materials 27, 3620-3625 (2015).

These facts are disclosed in order to illustrate the technical problem addressed by the present disclosure.

### GENERAL DESCRIPTION

The present disclosure relates to a plasmonic nanocarrier based system for light induced sequential release of multiple biomolecules. Gold nanorods (AuNR) modified with single stranded oligonucleotides were used for the attachment of biomolecules to the surface via DNA directed immobilization. Using DNA strands with distinct melting temperatures, the modulation of the release kinetics of two proteins with different light stimuli is achieved. Studies in SC-1 mouse fibroblasts show that part of the internalized AuNR are located inside the endosomes and that a laser irradiation at 780 nm promotes endosomal escape and the release of the proteins. Experiments with beta-galactosidase indicate that is possible to control the release of the protein while maintaining the enzymatic activity.

So far no formulation has the capacity to orchestrate the delivery of multiple proteins or microRNAs with remote control, as it is now disclosed. This is an important issue in many biological applications such as cell reprogramming. For example, lineage-switching experiments in the hematopoietic system have shown that the order in which two transcription factors become expressed in a progenitor can decide lineage outcome and thus timing is of utmost importance.

Advances in materials science have contributed for the development of systems that respond to external stimuli. In this regard, light-sensitive nanomaterials have emerged as an attractive solution to provide spatial-temporal control in the release of molecules within cells. A significant number of light triggerable formulations is constituted by polymers bearing photoresponsive groups that respond efficiently to UV light, which has enough energy to break a covalent bond or induce trans-cis isomerization. However UV light has a low penetration depth and may have cytotoxic effects. The application of photosensitizers in light responsive nanocarriers has also been described, but most of these respond to UV or visible light.

Gold nanorods (AuNRs) are controlled releasing agents in response to light excitation, due to their large optical cross section and their tuneable plasmon optical resonance in the near infrared range. The near infrared (NIR) plasmon resonance band is attractive for biomedical applications due the “water window” (650-900 nm) where there is low light absorbance by skin and tissue. Upon optical excitation, Au NRs show a strong photothermal response that is able to trigger a controlled release of DNA strands hybridized to complementary strands immobilized on the surface of the nanocarrier. Moreover, low intensity continuous wave irradiation facilitates endosomal escape of gold nanoparticles, thus increasing the therapeutic effect of the released biomolecule without affecting cell viability.

Although light triggered release of biomolecules has been reported, no formulation has achieved the photo-triggered release of multiple biomolecules in cells with temporal control.

The present disclosure relates to the development of an Au NR system for the delivery of biomolecules with spatio-temporal control using light as a trigger. Fluorescently labelled proteins were immobilized on the Au NR via DNA direct immobilization and using specific DNA strands for each protein. Release kinetics of both proteins were done upon laser irradiation, by monitoring the fluorescence in the suspension and supernatant. Mouse fibroblasts (SC-1) were incubated with NR modified with fluorescent proteins and submitted to different light stimuli. Total cell fluorescence, intracellular distribution of proteins and colocalization coefficients were determined. As a model enzyme, beta galactosidase was used. After incubation, the presence of the enzyme in SC-1 cells was confirmed by immunocytochemistry and its activity was determined using a commercially available kit (Abcam).

In an embodiment, AuNRs of dimensions [length, width], in particular 46.7, 13.7 nm, respectively, with the plasmon resonance wavelength at 780 nm were coated with ssDNA, where one strand of the DNA had a thiol moiety on its 5′ end, facilitating covalent attachment to the AuNR surface by an Au-thiol bond (FIG. 1). Proteins conjugated with the complement DNA sequence were then bound to its complementary DNA sequence immobilized at the nanoparticle surface (FIG. 1). For this work, two ssDNAs were designed that have a poly thymine spacer followed by an oligonucleotide sequence of 13 or 15 bases. Since proteins are bound to the AuNR surface via DNA hybridization, by changing the length and nucleotide composition of these sequences, it is possible to modulate the release of different proteins in a controlled and sequential manner. The poly-thymine spacer was used to keep the oligonucleotides in an upright position on the gold surface.

In an embodiment, in order to study the release profiles of proteins from gold NR, three groups of AuNR conjugates were prepared. Two of them were conjugated with DyLight-488 or DyLight 550 labelled BSA (DL₄₈₈-BSA or DL₅₅₀-BSA) separately and in one of them was conjugated the NR with both fluorescent proteins. Each of the AuNR formulation has a final amount of approximately 90 proteins. DL₅₅₀-dsDNA₆₈₉-AuNR- were used to test the protein release with three laser powers (0.8, 1.25 and 2 W/cm²).

In an embodiment, after 1 min of laser exposure, there are no significant differences between the three laser powers tested (FIG. 2a).

In an embodiment, after 2 min of irradiation, the fluorescent protein was only detected in the samples irradiated at 2 W/cm². The release of DL₄₈₈-BSA conjugated with DNA SEQ. ID. 1 (Tm 51.7° C.) was also tested with the lower laser power. In this embodiment, 2 min of irradiation were sufficient to release more than 60% of the conjugated protein (FIG. 2c). Consequently, in order to release both proteins independently from the same nanorod, the samples were irradiated for 2 min at 1.25 W/cm², releasing only one of the proteins and after centrifuging and resuspending again the pellet, the suspensions were irradiated for 3.5 min at 2 W/cm², which promoted the release of the other protein (FIG. 2d). In this case the release of the first protein with 2 min of irradiation at 1.25 W/cm² was superior to 90%, which is higher than the value achieved in the single protein system. This is attributed to a hindrance effect promoted by the other protein (DNA SEQ. ID. 2Tm 68.9° C.) which is not released with this stimulus and might be reducing the possibility of rehybridization of the released conjugate. Another explanation for this could be a change in the melting transition of the DNA strands in the dual protein system when compared to the single protein system.

In an embodiment, the selective release of DNA has been previously described using two different nanocarriers that responded to two different wavelengths, however the selective release of multiple proteins conjugated to the same nanoparticle has not been demonstrated yet. Moreover, so far there is no report of a light controllable system able to release selectively proteins inside cells.

The light induced release was also tested using beta-galactosidase as a model enzyme. For that, the protein was conjugated using DNA SEQ. ID. 1 (Tm 51.7° C.) as a linker. Although the release is achieved through a photothermal effect, it was detected enzymatic activity in the supernatant, which indicates that the protein denaturation is minimal.

Transfection of SC-1 cells with BSA-dsDNA_{51.7}-AuNR. The concentration of NR (50 μg/mL) and irradiation doses (1.25 to 2 W/cm², 2 min) used in cell studies do not show significant cytotoxic effects (FIG. 7).

In order to demonstrate the release of different proteins with different stimuli, cells were incubated with NRs bearing one or two fluorescent proteins (DL488-BSA and DL650-BSA). To monitor the intracellular localization of DL-BSA-dsDNA-AuNR, cells were washed with PBS and incubated for 15 min with lysotracker green (100 nM). The AuNRs used in experiments with cells were labelled with TRITC. Briefly, TRITC was bound to 1 kDa thiol-PEG-amine through the isothiocyanate group. Then, the fluorescent thiol-PEG was conjugated to the gold surface.

In FIG. 3a, irradiation with 1.25 or 2 W/cm² results in an increased fluorescence of the cell. This is an indication of the laser induced release of the protein, which results in less proximity between DL₄₈₈-BSA and AuNR with a consequent elimination of the quenching effect of the AuNR on the protein fluorescence. This increment in fluorescence correlates with the amount of protein released, indicating that higher laser powers lead to the release of more proteins.

In the non-irradiated cells, the overlap coefficient between AuNR and DL₄₈₈-BSA is not 100% (FIG. 3d). Since TRITC is in close proximity to the NR surface, its fluorescence might be quenched. This way, part of the AuNR fluorescence is not detected under confocal microscope, decreasing the overlap coefficient between DyLight and TRITC. Nevertheless, when the cells are irradiated, the colocalization between DL₄₈₈-BSA and AuNR decreases indicating that part of the protein was released from the AuNR.

With the application of two irradiation stimuli, the overlap coefficient between AuNR_{TRITC} and endosomes decreases (FIG. 3d). Although laser irradiation of gold NR leads to a localized thermal effect, the mechanism of endosomal escape is thought to be mediated by a photochemical process through the production of free radicals. Despite the evidence of endosomal escape promoted by the irradiation (supplementary FIG. 2), with the laser powers tested, this process is not 100% effective, thus part of the AuNR and the released proteins could be entrapped in the endosomes with a consequent colocalization under confocal microscope.

To investigate the intracellular delivery of two proteins within cells with temporal control, we have immobilized both DL₆₅₀-BSA-ssDNA_{51.7} and DL₄₈₈-BSA-ssDNA_{68.9} in AuNRs conjugated with TRITC followed by their incubation with cells for 4 h. Cells were then washed, activated with a NIR laser and fluorescence monitored by a confocal microscope. The first stimulus (1.25 W/cm², 2 min) induced primarily the release of DL₆₅₀-BSA-ssDNA_{51.7} while the second stimulus (2 W/cm², 2 min) induced primarily the release of DL₄₈₈-BSA-ssDNA_{68.9}. After the 2 stimuli the total of each protein released was lower than 50% likely due to the spatial confinement in the endolysosomal compartment and potential re-hybridization.

For the laser induced release, continuous wave (CW) excitation was used. CW leads to a local increase of temperature creating a gradient from the AuNR core to the bulk solvent, but does not cause cavitation or melting of the Au NR core. On the other hand, pulsed lasers are able to heat locally the AuNR core to high temperatures inducing melting and destabilization of the thiol Au bond. Although the use of pulsed lasers has been used to induce the cleavage of Au-thiol bonds releasing biomolecules such as siRNA, the selective release of cargos from the same nanocarrier is only achievable with CW lasers.

Transfection of SC-1 cells with AuNR-DNA-βGal. To demonstrate the ability of this nanocarrier to deliver a functional protein to cells, beta-galactosidase as a model enzyme used. The activity of the enzyme was determined using a beta-galactosidase Detection Kit (Abcam). The fluorescence of the x-Gal staining was detected by fluorescence microscopy. The corrected total cell fluorescence was quantified with ImajeJ and corrected for background fluorescence.

In cells incubated with AuNR-DNA-βGal (DNA SEQ. ID. 1-Tm 51.7° C.) there is a 1.5 fold increase in fluorescence after irradiation with a laser power of 1.25 W/cm². Using a higher laser power, the increase in fluorescence is less evident, probably due to the thermal degradation of the enzyme. However, in contrast with this, Krpetic and co-workers observed that laser irradiation with powers below 20 mW/cm² induces minimal temperature increase. When the activity test is performed one hour after laser activation, the enzymatic activity decreases (FIG. 10a). This means that beta-galactosidase might be benefiting from a protective environment against degradation by proteases while it is immobilized on the NRs. This protective effect is maintained when the time was extended between the end of the incubation with AuNRs and the laser activation (FIG. 10b). Moreover, although there is slight increase in the activity in cells treated with chloroquine, an inhibitor of lysosomal degradation, this difference is not statistically significant (FIG. 11). This also suggests that the immobilized beta-galactosidase is protected from degradation.

In FIG. 5a, the sum of enzymatic activity of the AuNR suspension after centrifugation and the supernatant is almost constant, indicating that the released and the immobilized proteins present the same level of enzymatic activity. The increase in activity in the irradiated cells is attributed to a change in the intracellular localization of the protein. With laser irradiation, the protein is able to escape the endosomes, which is confirmed by a lower overlap coefficient. Consequently, there is an increase in the environmental pH that has impact on the enzymatic activity (FIG. 12). Besides the increase in intensity, the fluorescence of X-Gal staining is also more spread throughout the cell as confirmed by a lower coefficient of variation (FIG. 5).

Beta-galactosidase was also detected by immunocytochemistry (FIG. 6). In cells incubated with AuNR-DNA-βGal it is observed an increase in fluorescence signal under confocal microscope. In the irradiated samples, the fluorescence is higher and spread in the cytosol, which suggests that the protein is released from the endosomes. When the cells were fixed one hour after activation, the amount of protein detected was lower, which correlates with a decreased enzymatic activity observed in the X-gal activity test performed under the same conditions.

In order to enable the immobilization of microRNAs on gold nanorods through hybridization of complementary DNA strands, miR-155-5p and miR-302a-3p modified with a terminal amine group were reacted with sulfo-GMBS and then with ssDNA containing a terminal thiol group (FIG. 13a). Each conjugate was purified by reverse-phase HPLC (FIG. 14)

The ssDNA-miR conjugates were hybridized at 37° C. using a 400 or 800-fold ratio of miR over AuNR concentration (FIG. 13b). Respectively, 202 and 390 DNA-miR-155 conjugates were immobilized per AuNR. Regarding DNA-miR-302a, the number of conjugates immobilized was 226 or 404 per AuNR.

The activity of each miR was monitored before and after conjugation with ssDNA. For that, miR-155, miR-302a, miR-155-ssDNA and miR-302a-ssDNA were complexed with lipofectamine RNAimax and then incubated with HEK-293T for 4 h or 24 h. In the concentration range tested (0.05 to 5 nM) the decrease in fluorescence correlates with the concentration of miR, i. e., a higher concentration accentuates the fluorescence decrease. At the highest concentration of miR-155 or miR-155-ssDNA (5 nM), mCherry fluorescence decreased almost 100% 60 h after transfection. In the case of miR-302a and miR-302a-ssDNA, a concentration of 5 nM was able to knockdown completely EGFP fluorescence 60 h after transfection. Both miRs are active after conjugation with ssDNA, although miR-302a-ssDNA conjugate is slightly less active than miR-302a at the lowest concentrations tested (0.05-0.5 nM) (FIG. 15c)

In order to study the laser induced release and activity of miR-DNA conjugates, it was used AuNRs hybridized with: I) miR-155 conjugated with ssDNA with a melting temperature of 51.7° C. and II) miR-302a conjugated with ssDNA with a melting temperature of 68.9° C. Each suspension of miR-dsDNA-AuNR was irradiated and immediately centrifuged. Then the supernatant was complexed for 20 min with lipofectamine RNAimax and HEK-293 cells were exposed to these complexes for 4 h and their fluorescence was monitored for 72 h. The amount of miR-155-ssDNA released with a laser stimulus of 1.25 W/cm2 for 2 min is able to induce 80% decrease in mCherry fluorescence (FIG. 16a). Increasing the time or the power of the laser does not decrease significantly mCherry fluorescence when compared to 2 min laser stimulus at 1.25 W/cm2. For miR302a-ssDNA conjugate, the release behaviour is different (FIG. 16b). Laser irradiation for 2 or 5 min at 1.25 W/cm2 induces no more than 12% decrease in EGFP fluorescence 72 h after transfection and more significant effects (up to 57% decrease) are only observed with higher laser power (2 W/cm2). Laser induced release of miR-DNA conjugates correlates with the power of the laser and the melting temperature of the DNA strands, i. e. miR conjugated with higher melting temperature DNA (68.9° C.) requires a higher laser power to be released from the AuNR surface.

The effect of cecropin-melittin on the uptake of miR-dsDNA-AuNR-TRITC was studied using confocal microscopy (FIG. 17a). The amount of miR-dsDNA-AuNR-TRITC that were internalized tends to increase with higher concentrations of cecropin-melittin used in the incubation, causing an increase in cell fluorescence (FIG. 17b). Moreover, lower colocalization with Lysotracker green was detected when cecropin-melittin was used. However, increasing the concentration of the peptide from 5 μM to 10 μM does not accentuate the decrease in colocalization (FIG. 17c).

The cytotoxicity of miR-dsDNA-AuNR co-incubated with cecropin-melittin and the effect of the irradiation were evaluated. For that, after incubation and irradiation, cells were left in the incubator for 24 h and then the ATP was measured using Celltiter-Glo Luminescent Cell Viability Assay (Promega). At 10 μM, cecropin-melittin only causes 8% decrease in ATP production. Moreover, in all the concentrations tested, there is no significant effect of the irradiation in cell viability (FIG. 18).

To test the laser induced release of miR-ssDNA conjugates in cells, HEK-293 were incubated for 4 h with miR155-dsDNA_{51.7}-AuNR or miR302a-dsDNA_{68.9}-AuNR (50 μg/mL). Cells were then irradiated for 2 min with different laser powers (1.25 and 2 W/cm²). In line with what was observed in the assay performed with lipofectamine (FIG. 16), each formulation responded differently to the stimuli. In cells incubated with miR155-dsDNA_{51.7}-AuNR, laser stimuli of 1.25 W/cm² or 2 W/cm² are able to reduce mCherry fluorescence in more than 60% after 72 h and there is no difference between each group (1.25 W/cm² and 2 W/cm²) (FIG. 19a). On the other hand, in cells incubated with miR302a-dsDNA_{68.9}-AuNR, irradiation at 2 W/cm² is significantly more efficient in releasing miR302a and inducing EGFP knockdown than irradiation at 1.25 W/cm² (FIG. 19b).

For the sequential release of miR-155 and miR-302a, cells were incubated with miR155-dsDNA_{51.7}-AuNR (25 μg/mL) and miR302a-dsDNA_{68.9}-AuNR (25 μg/mL) for 4 h. After incubation, cells were irradiated for 2 min at 1.25 W/cm². A second stimulus (2 min, 2 W/cm²) was applied 2 h after the first stimulus. miR-155, which is conjugated to the oligonucleotide with lower melting temperature (Tm 51.7° C.), was preferentially released with the first stimulus. The release of miR-155 correlates with a 42% decrease in mCherry fluorescence 48 h after the stimulus (FIG. 20a). On the other hand, miR-302a which is conjugated to the oligonucleotide with higher melting temperature oligonucleotide (Tm 68.9° C.), is not released with irradiation at 1.25 W/cm², thus, no significant decrease in EGFP was observed. Knockdown of EGFP (41% decrease in EGFP 48 h after irradiation) was only detected when a higher energy stimulus (2 min, 2 W/cm²) was applied (FIG. 20b).

The present disclosure describes a composition for controlled release of a biomolecule comprising:
a gold nanoparticle;
at least two different biomolecule;
at least two different oligonucleotides sequences as a binder, comprising different melting points,
wherein the oligonucleotides sequences binds the biomolecules to the surface of the gold nanoparticle;
wherein each oligonucleotide is a photo-active molecular agent releasing the biomolecule by photo-activation.

The composition of the present disclosure may be use in medicine, namely in the treatment of cancer, aging diseases, and accelerated aging syndromes, infectious diseases such as AIDS, epigenetic diseases such as polycystic ovary syndrome, or cardiac diseases, such as cardiac regeneration.

In an embodiment, biomolecule may be selected from a list consisting of: a peptide, a protein a microRNA, a mRNA, and mixtures thereof.

In an embodiment, disclosure relates to a composition for controlled release of peptides, proteins or microRNAs comprising:
a gold nanoparticle;
at least two different biomolecules;
at least two different oligonucleotides having different melting points, binding said biomolecules to the surface of the gold nanoparticle;
wherein the binding is obtainable via hybridization of complementary oligonucleotides;
wherein each of the oligonucleotides is a photo-active such that the respective biomolecule is releasable by photo-activation.

In an embodiment, the oligonucleotide sequences may comprise between 13-30 base pairs, preferably 13-16 base pairs.

In an embodiment, the oligonucleotide sequences may further comprise a poly thymine spacer.

In an embodiment, the melting point of a oligonucleotide sequence of the plurality of oligonucleotides sequences varies between 40-90° C., preferably between 50-75° C., more preferably 51.7-68.9° C.

In an embodiment, a first oligonucleotide sequence of the plurality of oligonucleotides sequences has a melting point of 51.7, and a second oligonucleotide sequence of the plurality of oligonucleotides sequences has a melting point of 68.9° C.

In an embodiment, the oligonucleotide sequences further comprise a poly thymine spacer.

In an embodiment, the gold nanoparticle may be a nanorod.

In an embodiment, the protein may be attached to the gold nanoparticle by DNA hybridization.

In an embodiment, the composition previously described may comprise at least two oligonucleotides, wherein each oligonucleotide is a sequence 95% identical to:

SEQ. ID 1:
5′-HS-C6-TTTTTTTTTTTTTTTATAACTTCGTATA-3′;
SEQ. ID 2:
5′-HS-C6-TTTTTTTTTTTTGTCCGGGTCCAGGGC-3′;
SEQ. ID 3:
5′-HS-C6-TATACGAAGTTATAAAAAAAAAA-3′;
SEQ. ID 4:
5′-HS-C6-TGCCCTGGACCCGGAC-3′;

or mixtures thereof.
in particular identical to SEQ. ID 1, SEQ. ID 2, SEQ. ID 3, SEQ. ID 4, or mixtures thereof.

In an embodiment, the composition previously described may comprise two oligonucleotides, wherein each oligonucleotide is a sequence 96% identical to: SEQ. ID 1, SEQ. ID 2, SEQ. ID 3, SEQ. ID 4, or mixtures thereof, in particular at least 97% identical, at least 98% identical, at least 99% identical, or mixtures thereof.

In an embodiment, the composition previously described may comprise at least an oligonucleotides sequence identical to: SEQ. ID 1, SEQ. ID 2, SEQ. ID 3, SEQ. ID 4, or mixtures thereof.

In an embodiment, the composition previously described may comprise comprising a plurality of oligonucleotides sequences are SEQ. ID 1 and SEQ. ID 2, or SEQ. ID 3 and SEQ. ID 4.

In an embodiment, methods for the alignment of sequences for comparison are well known in the art, such methods include GAP, BESTFIT, BLAST. GAP uses the algorithm of Needleman and Wunsch ((1970) J Mol Biol 48: 443-453) to find the global (over the whole the sequence) alignment of two sequences that maximizes the number of matches and minimizes the number of gaps. The BLAST algorithm (Altschul et al. (1990) J Mol Biol 215: 403-10) calculates percent sequence identity and performs a statistical analysis of the similarity between the two sequences. The software for performing BLAST analysis is publicly available through the National Centre for Biotechnology Information (NCBI). Homologues may readily be identified using, for example, the ClustalW multiple sequence alignment algorithm (version 1.83), with the default pairwise alignment parameters, and a scoring method in percentage. Global percentages of similarity and identity may also be determined using one of the methods available in the MatGAT software package (Campanella et al., BMC Bioinformatics. 2003 Jul. 10; 4:29. MatGAT: an application that generates similarity/identity matrices using protein or DNA sequences). Minor manual editing may be performed to optimise alignment between conserved motifs, as would be apparent to a person skilled in the art. Furthermore, instead of using full-length sequences for the identification of homologues, specific domains may also be used.

In an embodiment, the composition of the present disclosure may further comprises a cell transfection enhancer, preferably penetrating peptide, in particular cecropin melittin.

In an embodiment, the protein can be albumin, transcription factor, enzyme.

In an embodiment, the density of oligonucleotide per gold nanoparticle is between 10-400. Preferably, the amount of oligonucleotides per AuNR may be determined indirectly in the supernatant by measuring absorbance at 260 nm in Nanodrop.

In an embodiment, the gold nanoparticle has a size between 10-45 nm, preferably a size between 15-30 nm.

In an embodiment, the gold nanoparticle has aspect ratio of 1.5-10, preferably between 3-3.4. Preferably, the aspect ratio may be given by a.r.=L/W, where L is the length of the AuNR and W is the width of the AuNR. The length and the width were determined by analysis of transmission electron microscopy images.

In an embodiment, the determination of gold nanoparticle size may be achieved by transmission electron microscopy.

In an embodiment, the composition of the present disclosure may be a topic formulation or an injectable formulation.

In an embodiment, the composition of the present disclosure may be for the use in medicine.

In an embodiment, the composition of the present disclosure may be for the use in regenerative medicine.

In an embodiment, the composition of the present disclosure may be for the use in cell reprograming, in particular adult stem cells, and/or embryonic stem cells.

In an embodiment, the composition of the present disclosure may be use in the treatment of cancer, aging diseases, and accelerated aging syndromes, infectious diseases such as AIDS, epigenetic diseases such as polycystic ovary syndrome, or cardiac diseases, such as cardiac regeneration.

Another aspect of the present disclosure is the use of the composition of the present subject-matter as a coating of medical devices wherein the composition further comprises an adhesive to immobilize the nanoparticles on top of the medical device.

In an embodiment, the medical device may be a patch, a catheter, a stent, a contact lens or a pacemaker.

In an embodiment, the composition of the present disclosure may be for the use in the treatment of diseases that respond positively to cell reprograming.

In an embodiment, the composition of the present disclosure may be for the use in the treatment of retinal diseases, neurodegenerative diseases, or viral infections.

Throughout the description and claims the word “comprise” and variations of the word, are not intended to exclude other technical features, additives, components, or steps. Additional objectives, advantages and features of the solution will become apparent to those skilled in the art upon examination of the description or may be learned by practice of the solution.

### BRIEF DESCRIPTION OF THE DRAWINGS

The following figures provide preferred embodiments for the present disclosure and should not be seen as limiting the scope of the disclosure.

FIG. 1: Schematic representation of AuNR-DNA-protein. A DNA-protein conjugate is bound to the AuNR surface through hybridization with a complementary DNA strand attached to the AuNR via thiol gold chemistry. Light induced sequential release of different proteins from the AuNR upon the application of different stimuli.

FIG. 2: Light induced release profiles of DL-BSA attached to gold nanorods. a) Effect of the laser power in the amount of protein released b) Percentage of protein in the supernatant and attached to the NR after irradiation c) Effect of the melting temperature of the DNA on the amount of BSA released from the NR. Quantifications were done by measuring the fluorescence of DL-BSA (n=3). d) Laser induced sequential release of DL₄₈₈-BSA and DL₅₅₀-BSA from AuNR. A suspension of DL_{488;550}-BSA-dsDNA-AuNR-BSA was first irradiated for 2 min at 1.25 W/cm² and then centrifuged in order to collect the supernatant. The AuNR were resuspended and irradiated again at 2 W/cm² for 3.5 min and then centrifuged. The fluorescence of both supernatants was measured in a Biotek HT plate reader.

FIG. 3: A) Confocal images of SC-1 cells incubated with DL₄₈₈-BSA-dsDNA-AuNR_{TRITC} (50 μg/mL; DNA Tm 68.9° C.) without and with laser irradiation (1.25 W/cm² and 2 W/cm² for 2 min). b and c) Intensity and coefficient of variation of the signal of BSA-DL₄₈₈. d) colocalization between AuNR_{TRITC} and DL488-BSA.

FIG. 4: Intracellular delivery of two proteins. Fibroblasts were incubated with AuNR-TRITC conjugated with DL650-BSA-dsDNA51.7 and DL488-BSA-dsDNA68.9 for 4 h. Cells were then washed with cell culture media and irradiated for 2 min at 1.25 W cm⁻². A subset of samples was fixed with 4% PFA after irradiation and the other group was incubated for additional 5 min before being irradiated for 2 min at 2 W/cm² and fixed afterwards. The amount of protein released was calculated as % BSAR=100×(MCbefore−MCafter)/MCbefore, where MCbefore is the Manders' colocalization coefficient before irradiation and MCafter is the Manders' colocalization coefficient after irradiation. Results are Average±SEM, n=3 (3 samples, 5 microscope fields per sample). Unpaired t-test was used to compare each condition (p value≤0.0001).

FIG. 5: Activity of beta-galactosidase in SC-1 after 4 h incubation with βGal-dsDNA_{51.7}AuNR (50 μg/mL) After irradiation, the activity of beta-galactosidase was determined with a beta galactosidase Detection Kit (Abcam). Briefly, the cells were fixed incubated overnight with X-Gal substrate and then observed in confocal microscope using 633 nm laser excitation. A) Fluorescence of Xgal staining assessed by confocal microscopy. B) corrected total cell fluorescence of SC-1 cells incubated with Xgal substrate after incubation with βGal-dsDNA_{51.7}AuNR and irradiation with different laser powers. C) coefficient of variation of the xGal staining.

FIG. 6: Distribution of beta-galactosidase assessed by immunocytochemistry. Confocal images of SC-1 cells incubated with βGal-dsDNA_{51.7}AuNR (50 μg/mL) with and without laser irradiation. After laser treatment the cells were fixed and immunostained using a rabbit anti-beta galactosidase primary antibody and anti-rabbit alexa-fluor 488 as secondary antibody.

FIG. 7: Cytotoxicity of BSA-dsDNA_{51.7}-AuNR. Mouse fibroblasts were seeded on a 96 well plate (4×10³ cells/well), left to adhere for 24 h and then incubated with different concentrations of BSA-dsDNA_{51.7}-AuNR for 4 h. After incubation, the cells were washed with medium to remove non-internalized AuNR. Some of the conditions were irradiated for 2 min with 1.25 or 2 W/cm². Then, the plate was left in the incubator for 24 h. The ATP production was measured by a Celltiter-Glo Luminescent Cell Viability Assay (Promega).

FIG. 8: Colocalization between calcein and lysotracker red in SC-1 cells incubated with BSA-dsDNA_{51.7}-AuNR. Cells were incubated with 50 μg/mL of BSA-dsDNA_{51.7}-AuNR and 25 mM calcein for 4 h. After replacing the medium, cells were incubated with lysotracker red (100 nM) for 15 min and then irradiated for 2 min with 1.25 or 2 W/cm² and observed in confocal microscope.

FIG. 9: Enzymatic activity of beta-galactosidase followed by absorbance at 420 nm. After laser irradiation with 0.57 W/cm², NR suspension was centrifuged at 9000 g, the supernatant was collected and the pellet was resuspended in 10 mM phosphate buffer. Then, 50 μL of supernatant or suspension were added to 100 μL of ONPG (13 mg/mL) prepared in 0.1 M phosphate buffer The absorbance at 420 nm was measured for 30 min at 37° C. in a 96 well plate using a Synergy HT microplate reader. The slope of the curves was used to calculate the amount of active protein.

FIG. 10: Activity of beta-galactosidase in SC-1 cells after 4 h incubation with βGal-dsDNA_{51.7}AuNR (50 μg/mL). a) effect of the time between laser activation and the activity assay. B) influence of the time between the end of incubation and laser activation.

FIG. 11: Activity of beta-galactosidase in SC-1 mouse fibroblasts after 4 h incubation with βGal-dsDNA_{51.7}AuNR (50 μg/mL) in the presence of 100 μM chloroquine. After irradiation, the activity of beta-galactosidase was determined with a beta galactosidase Detection Kit (Abcam).

FIG. 12: Enzymatic activity of beta-galactosidase followed by absorbance at 420 nm. 50 μL of beta galactosidase solutions (0.4 μg/m L) prepared in 0.1 M phosphate buffer (pH 6.0; 6.8; 7.0) were added to 100 μL of ONPG (13 mg/mL) also prepared in 0.1 M phosphate buffer (pH 6.0; 6.8; 7.0). The absorbance at 420 nm was measured for 30 min at 37° C. in a 96 well plate using a Synergy HT microplate reader.

FIG. 13: Preparation of miR-dsDNA-AuNR conjugates. (a) Preparation of ssDNA-miR conjugates. miR-155 or miR302a were initially reacted with a heterofunctional linker (Sulfo-GM BS) by its terminal succinimide ester. The miR conjugate was then reacted with a ssDNA having a terminal thiol group. After reaction, the miR conjugate (miR155-ssDNA or miR302a-ssDNA) was purified by HPLC. (b) Preparation of AuNR-ssDNA. AuNRs were reacted with HS-ssDNA complementary to the strands of miR155-ssDNA or miR302a-ssDNA conjugates. The miR-ssDNA conjugates were then bound to the ssDNA-AuNR by hybridization. The surface of AuNR was then filled with thiol-PEG. Upon NIR irradiation, there is an increase in the temperature at the AuNR leading to the DNA de-hybridization and the release of miRs with different kinetics. The release kinetic depends on the heat generated (which depends on the power of NIR laser used) and the melting temperature of the oligonucleotides.

FIG. 14: Characterization of miR-ssDNA conjugates. Chromatograms of the HPLC purification of a) miR-155-ssDNA and b) miR-302a-ssDNA. c) and d) Characterization of HPLC fractions by electrophoresis. Lines a, b and c are relative to the reaction mixture, control miR and control ssDNA respectively. Lines 1-5 are relative to the HPLC fractions represented in each chromatogram.

FIG. 15: a) Fluorescence microscopy images of hEK-293T cells transfected with miR-ssDNA conjugates. Images correspond to 24 h transfection with 2.5 nM miR-ssDNA conjugates (60 h after transfection). Scale bar corresponds to 50 μm. b and c) HEK.293T cells were transfected with lipofectamine RNAimax and miR-155-ssDNA or miR-302a-ssDNA. Cells were exposed to the conjugates for 4 h or 24 h and their fluorescence was monitored afterwards in a high-content fluorescence microscope. EGFP and mCherry fluorescence were normalized to the control (cells transfected with scramble miR).

FIG. 16—EGFP and mCherry knockdown in HEK-293T after laser induced release of miR-ssDNA conjugates. a) mCherry fluorescence of cells transfected with supernatants from irradiated suspension of miR-155-dsDNA51.7-AuNR. b) EGFP fluorescence of cells transfected with supernatants from irradiated suspension of miR-302a-dsDNA_{68.9}-AuNR. Briefly, suspensions of miR-155-dsDNA51.7-AuNR and miR-302a-dsDNA_{68.9}-AuNR (20 μg/mL in 10 mM phosphate buffer pH 7.4 with 30 mM NaCl) were irradiated with different laser powers and times of irradiation and immediately centrifuged. Then the supernatants were complexed for 20 min with lipofectamine RNAimax (35 μL of supernatant complexed with 35 μL of RNAimax diluted 1:50 in DMEM) and 20 μL of this mixture was added to each well containing 100 μL of DMEM with 10% FBS. After incubation, cells were kept in DMEM (10% FBS, 0.3 μg/mL of Hoechst) and their fluorescence was monitored in a high-content fluorescence microscope (IN Cell 2200, GE Healthcare)

FIG. 17—a) Confocal images of cells stained with lysotracker green after 4 h incubation with miR-155-dsDNA-AuNR-TRITC (50 μg/mL) and cecropin-melittin. Scale bar is 30 urn. b) Intensity of the signal of AuNR-TRITC. c) Colocalization between AuNR-TRITC and lysotracker green expressed as Manders' overlap coefficient assessed by Image) analysis. In b and c, the results are expressed as Average±SD (n=3). *** denotes statistical significance (p<0.001) assessed by one-way ANOVA followed by Tukey's post-hoc test.

FIG. 18—Cytotoxicity of DNA51.7-AuNR. HEK-293T cells were incubated with DNA51.7-AuNR (50 μg/mL) without or with cecropin-melittin (5 and 10 μM) for 4 h. Then cells were washed and new medium was added (DMEM with 10% FBS). Subsequently, cells were irradiated for 2 min at 1.25 W/cm² and left in the incubator. After 2 h, cells were irradiated for 2 min at 2 W/cm² and then incubated for additional 24 h at 37° C. Cell metabolism was evaluated by an ATP assay.

FIG. 19—EGFP and mCherry knockdown after laser induced release of miR-ssDNA conjugates in HEK-293T. a) Normalization of mCherry fluorescence relative to EGFP fluorescence in cells incubated for 4 h with miR-155-dsDNA_{51.7}-AuNR with and without laser irradiation. b) Normalization of EGFP fluorescence relative to mCherry fluorescence in cells incubated for 4 h with miR-302a-dsDNA_{68.9}-AuNR with and without laser irradiation. Cell fluorescence was monitored in a high-content fluorescence microscope.

FIG. 20—a) and b) Fluorescence microscopy images of cells incubated with miR-155-dsDNA-AuNR and miR-302a-dsDNA-AuNR for 4 h in the presence of cecropin melittin (10 μM). After incubation cells were exposed to one laser stimulus (2 min at 1.25 W/cm²) or two laser stimuli (2 min at 1.25 W/cm² and 2 min at 2 W/cm²) with an interval of 2 h between each stimulus. c) and d) quantification of cell fluorescence 48 h after laser irradiation.

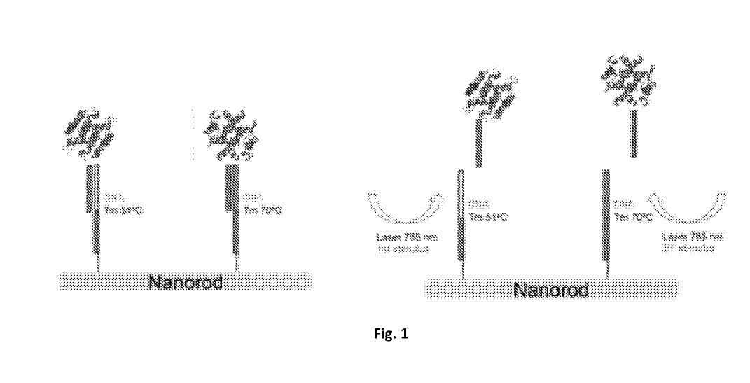

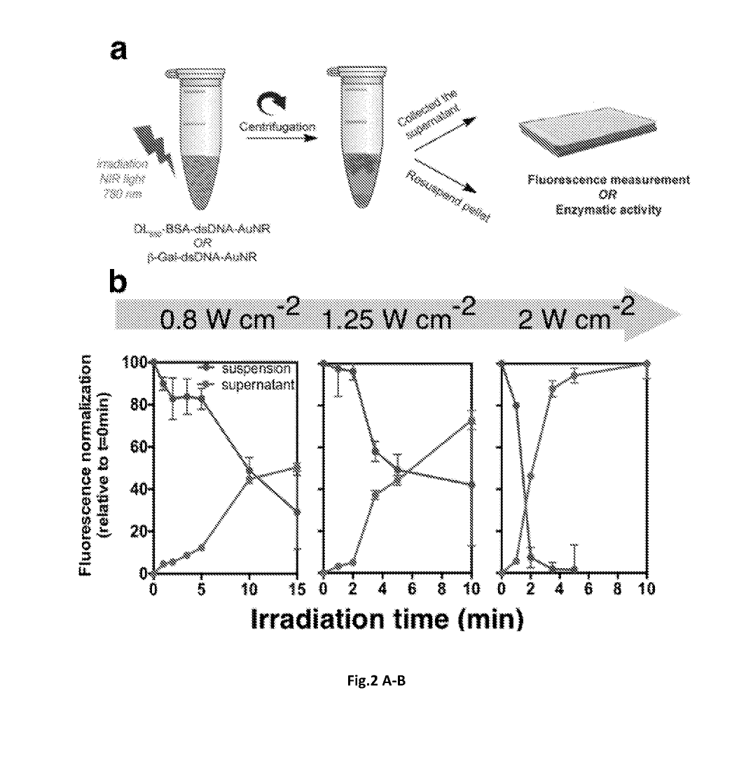

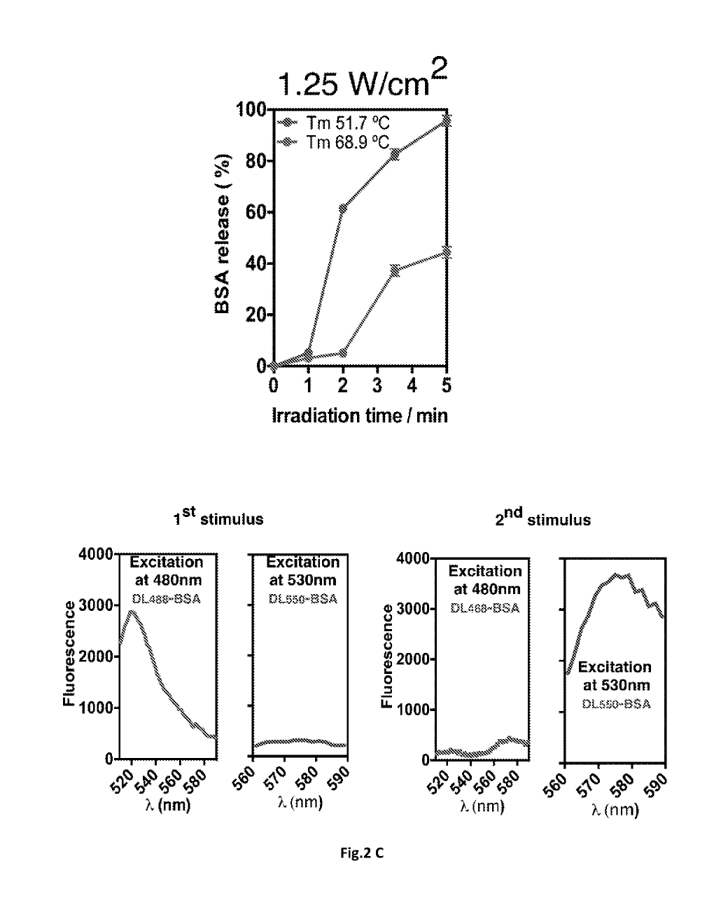

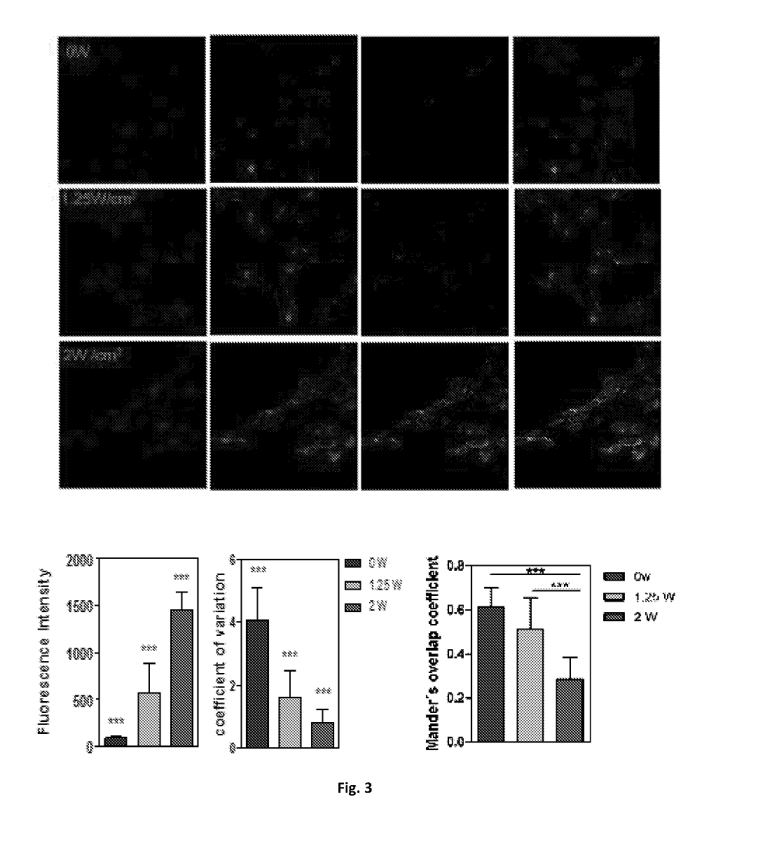

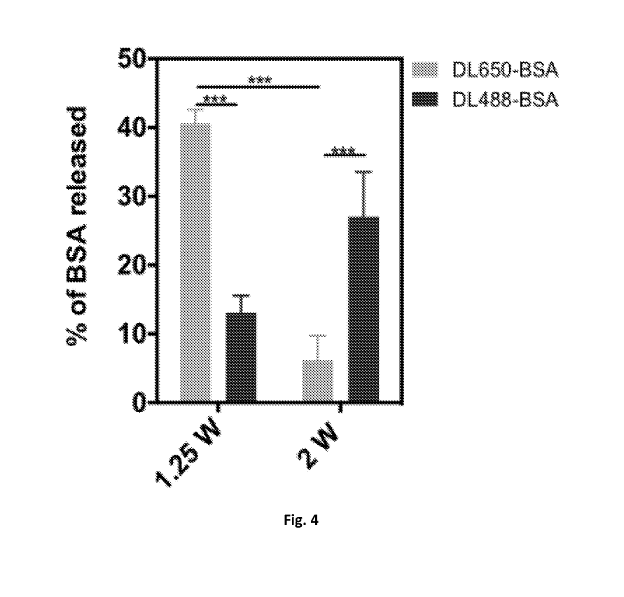

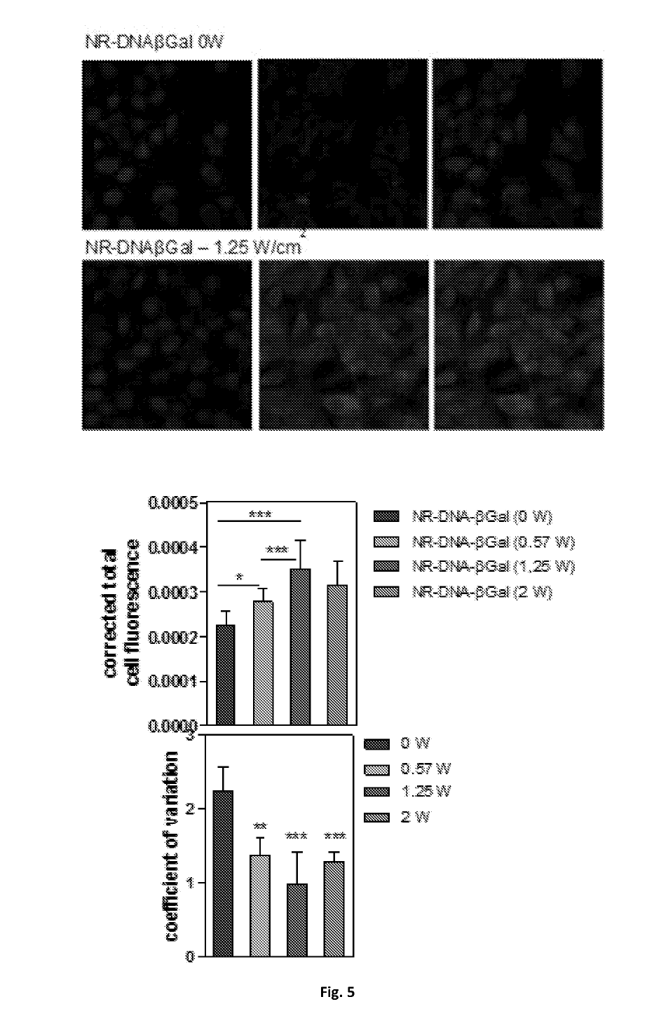

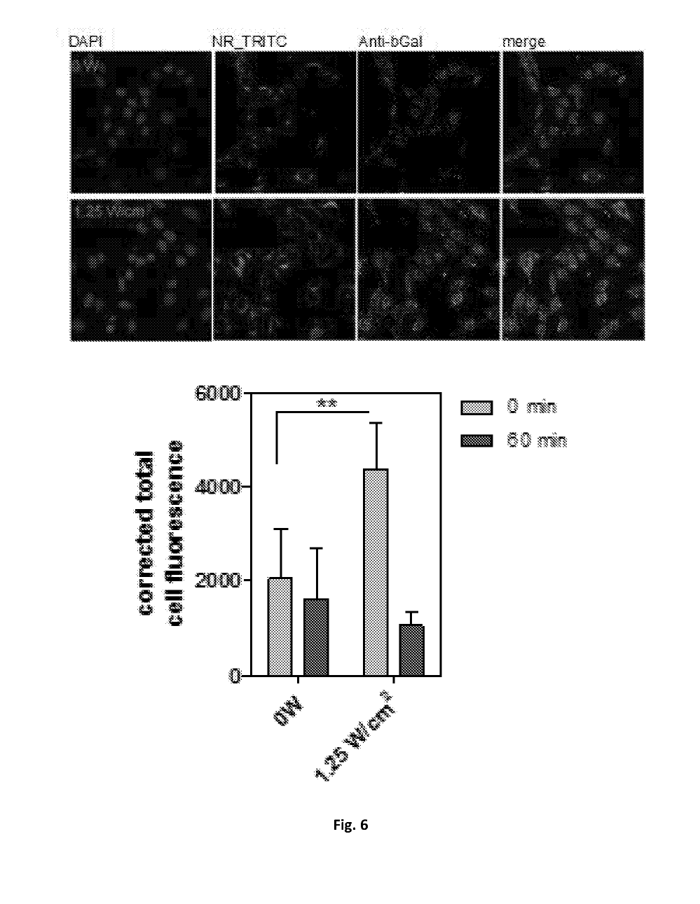

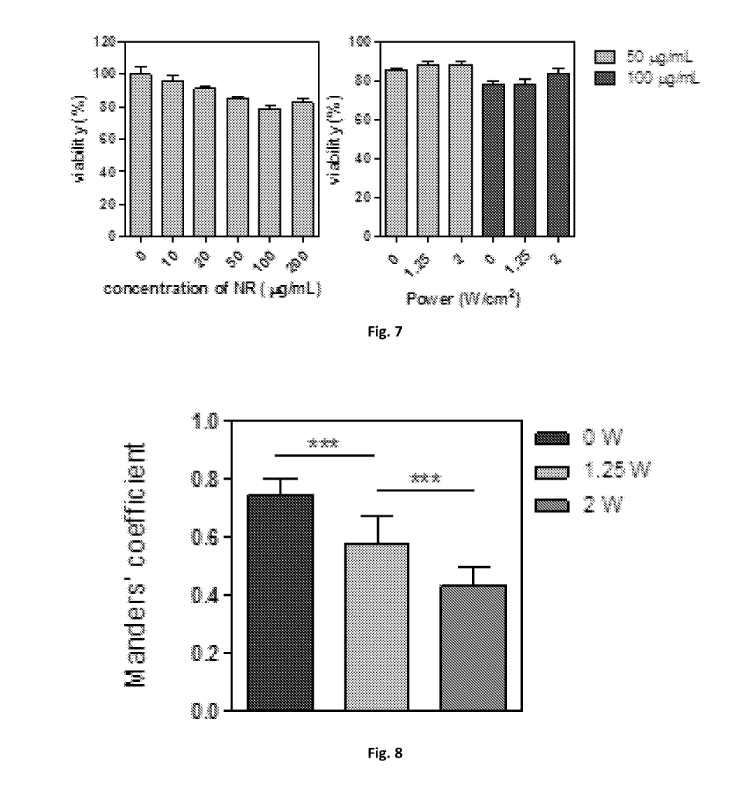

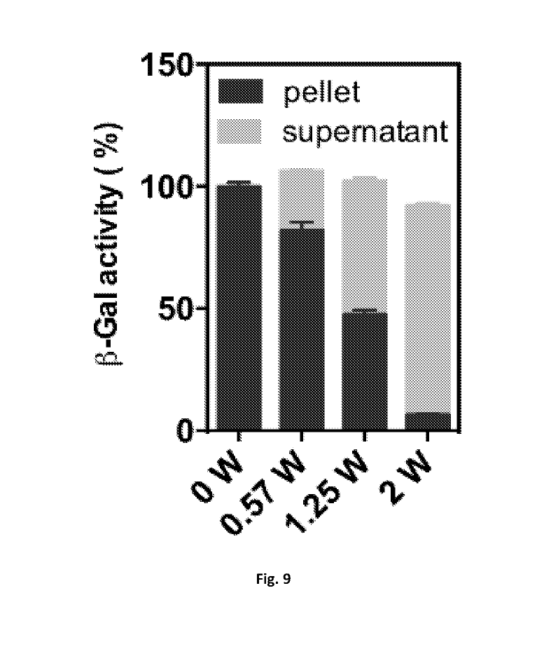

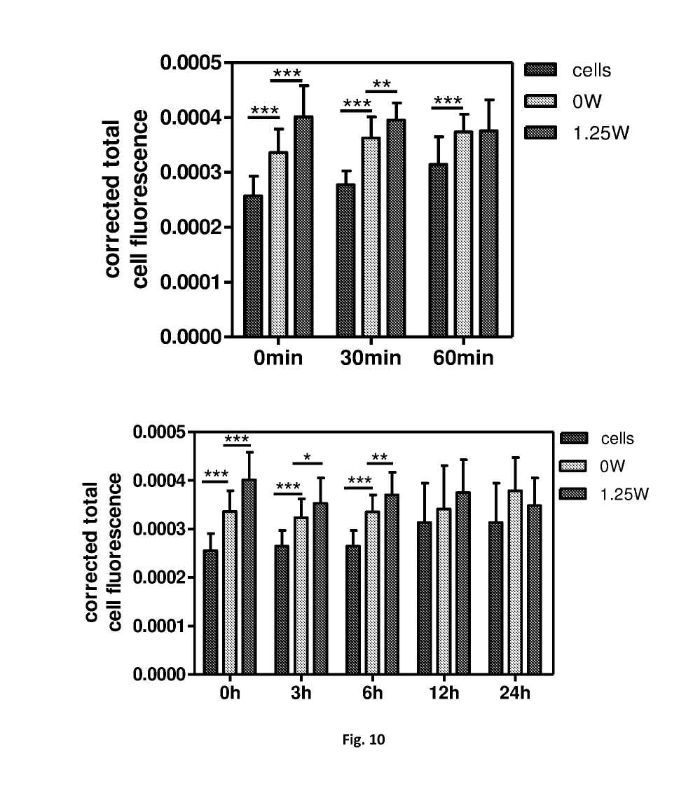

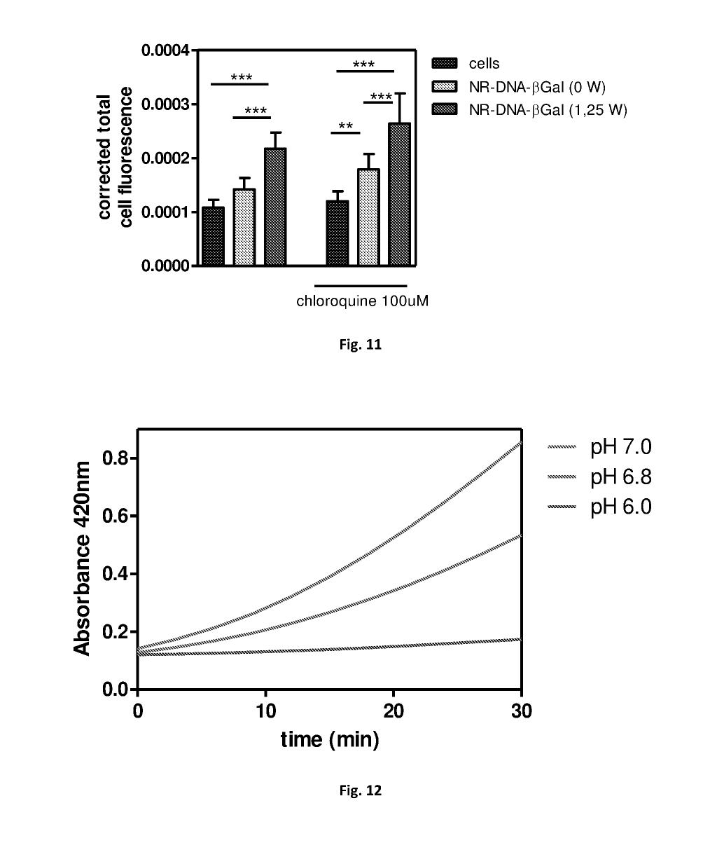

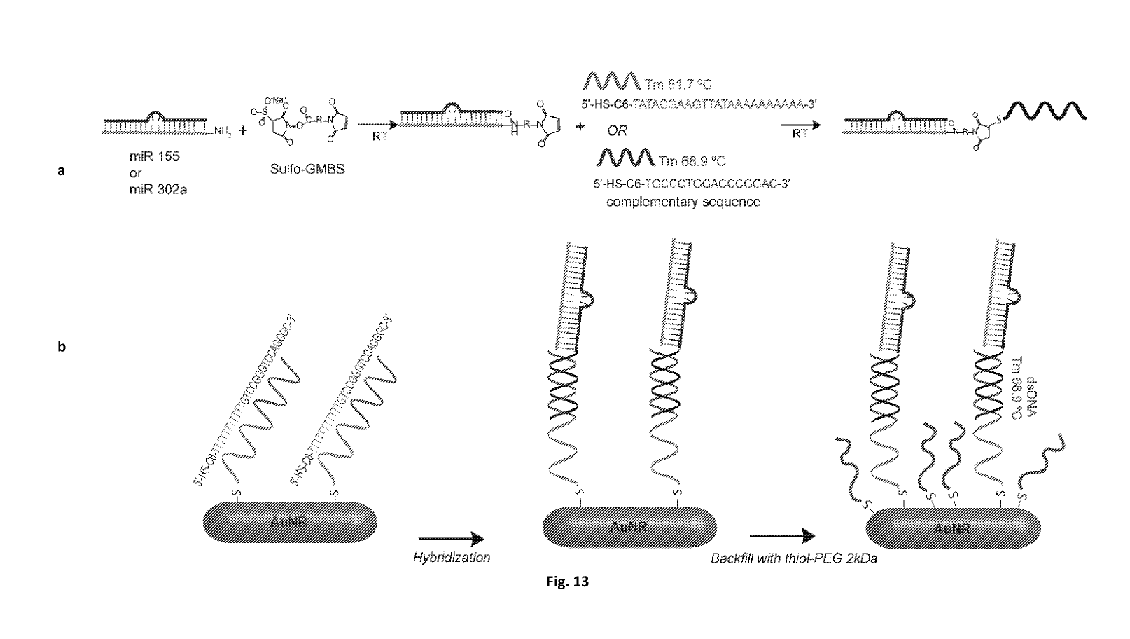

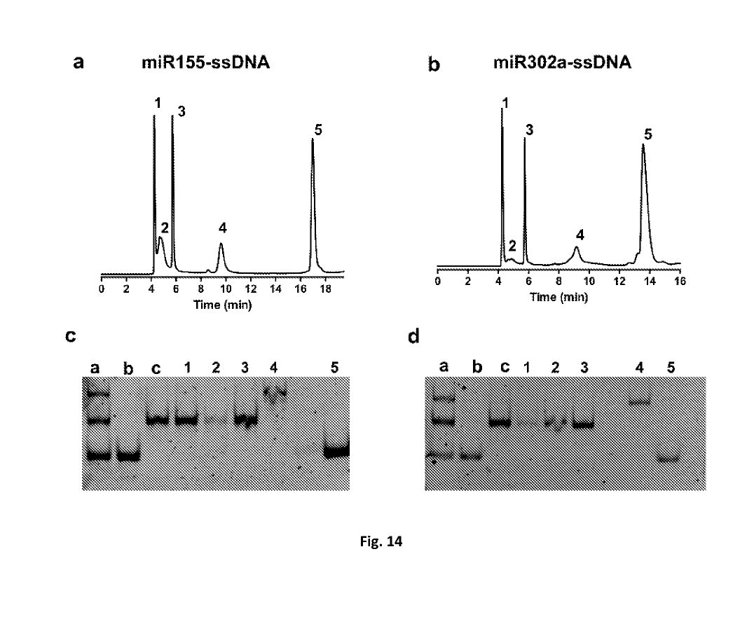

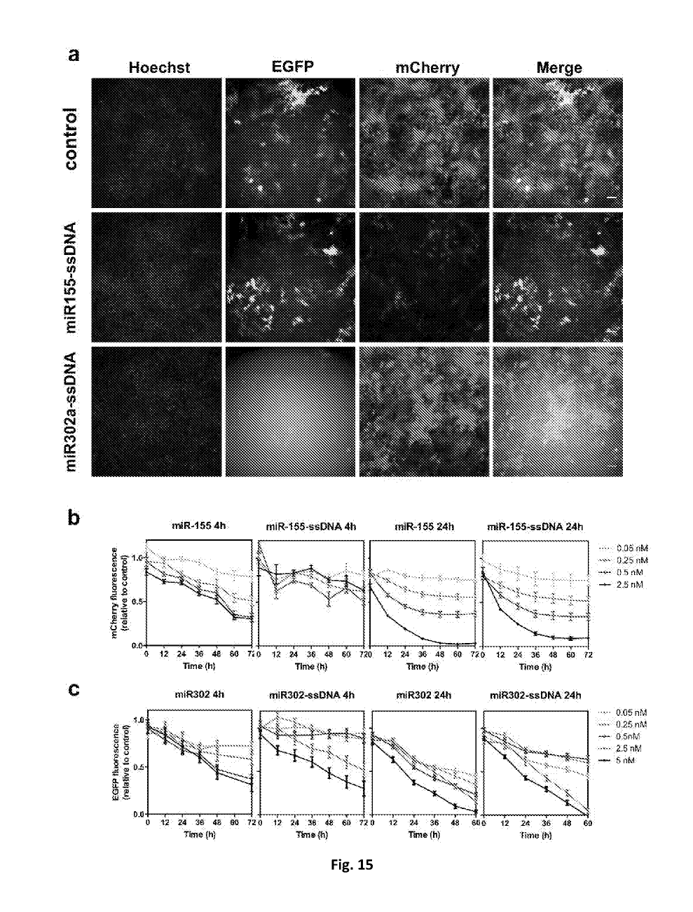

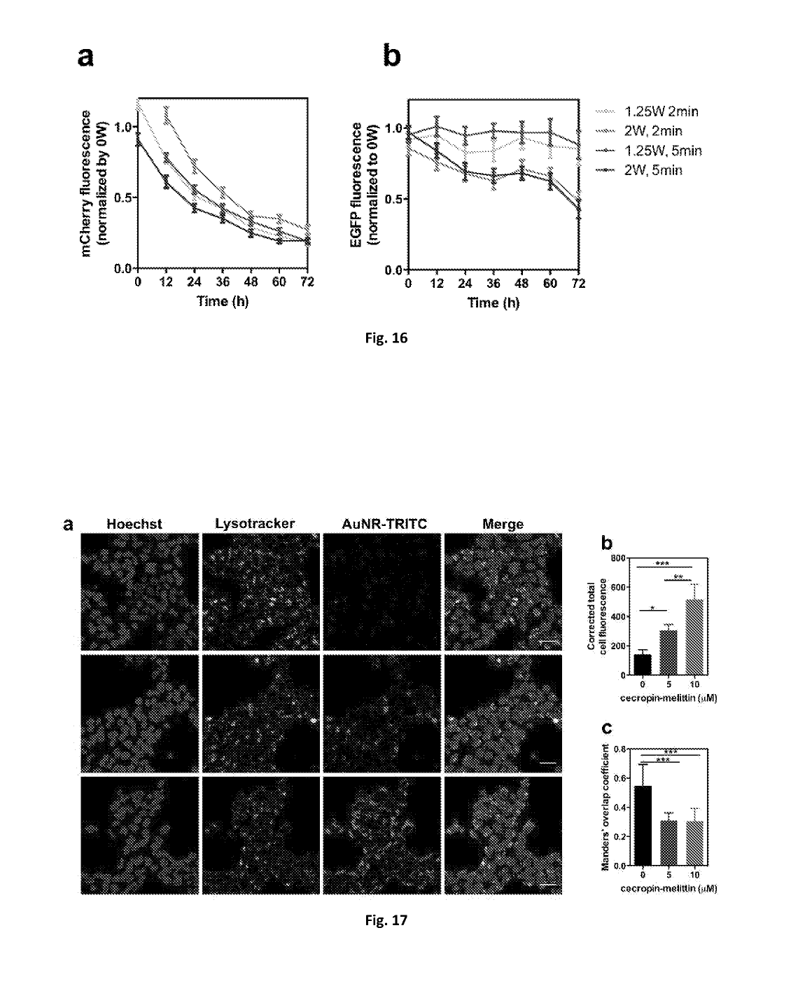

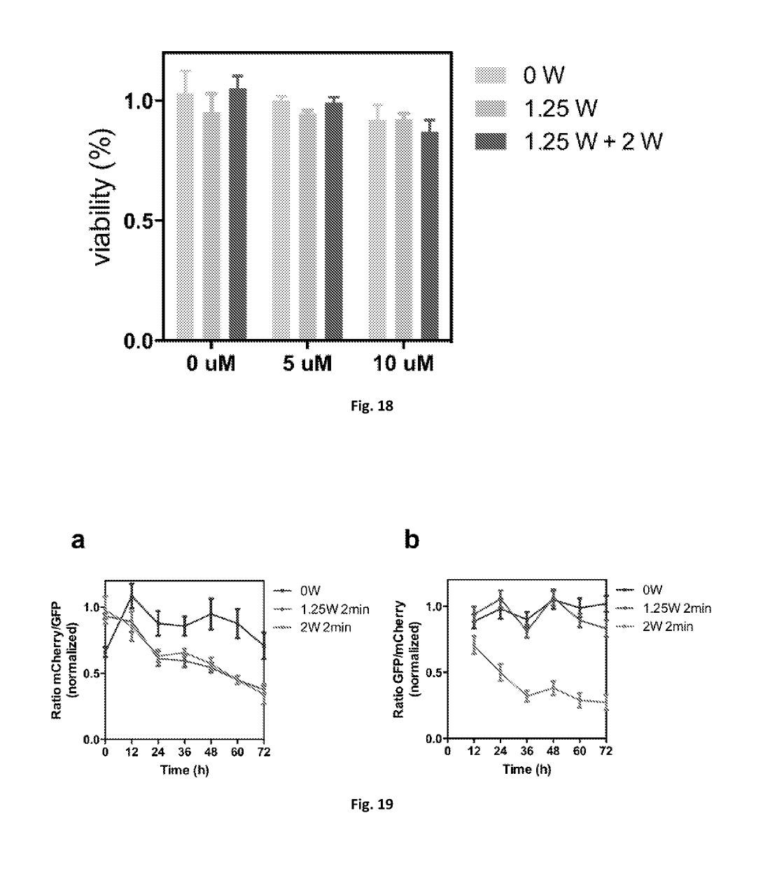

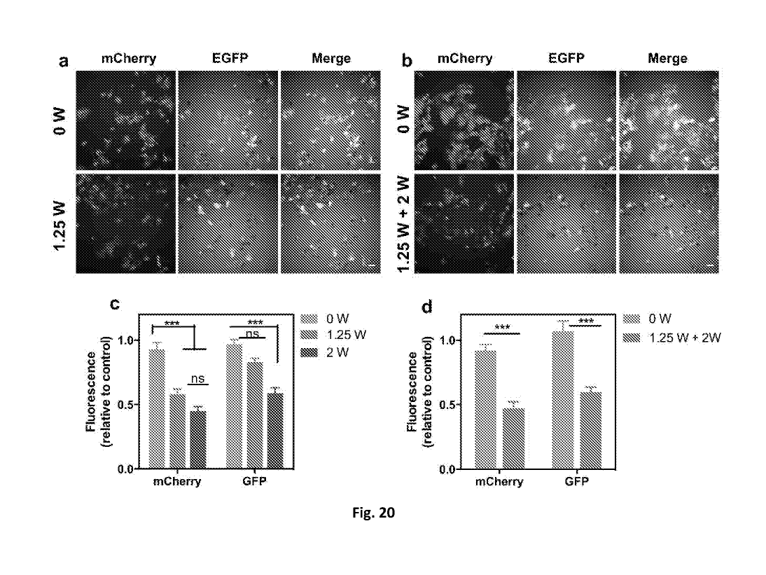

The present disclosure relates to the modulation of cell activity the sequential release of several proteins from a given nanocarrier using a wavelength, in particular this disclosure relates with a system that is able to release sequentially several proteins from the same nanocarrier using only one wavelength and just varying the laser power or exposure time. Moreover, the formulation as well as the stimuli used for the controlled release are not cytotoxic. Finally, proteins immobilized on the NR surface are protected from degradation and light stimulation does not decrease their activity.

Au NR synthesis. Au NRs were prepared using the seed mediated method. For the preparation of the seed solution, chloroauric acid (HAuCl₄) (0.1 M, 12.5 μL) was added to a hexadecyltrimethylammonium bromide (CTAB) solution (0.1 M, 5 mL) and stirred vigorously for 5 min, after which an ice-cold sodium borohydride (NaBH4) (10 mM, 0.3 mL) was added. After stirring for 2 min the solution was kept at 25° C. For the preparation of growth solution of silver nitrate (AgNO₃) (5 mM; 3.2 mL) was added to CTAB solution (0.1 M; 200 mL) and mixed gently, after which HAuCl₄ (50 mM, 2 mL) was added. After mixing, ascorbic acid (0.1 M; 1.5 mL) was added. The solution changed from dark yellow to colourless. Finally, 1.5 mL of the seed solution (aged for 8 min at 25° C.) was added to the growth solution. The solution was kept at 28° C. for 2 h. The NRs were washed by centrifugation at 8.500 rpm and resuspended in water.

The CTAB on the NR surface was replaced using a method already reported with some modifications, in particular hexanethiol (1.5 mL) was added to the NR-CTAB suspension (1 mL; 2.5 nM). Then, acetone (3 mL) was added and the mixture was swirled for a few seconds. The aqueous phase became clear indicating ligand exchange and the organic phase containing the NRs was extracted. Then, a mixture of toluene (2 mL) and methanol (5 mL) was added to the organic phase. The solution was centrifuged at 5.000 g, 10 min, and the pellet was resuspended in 0.5 mL of toluene by brief sonication. The organic to aqueous phase was performed as follows. NR-hexanethiol (1 mL) in toluene was added to 9 mL of mercaptohexanoic acid (MHA; 5 mM; 9 mL) in toluene at 95° C. The reaction proceeded under reflux with magnetic stirring for 15 min. The precipitation of NRs indicated successful coating by MHA. After cooling to room temperature, the aggregates were washed twice with toluene by decantation. Finally, the NRs were washed with isopropanol to deprotonate the carboxylic groups and then the aggregates were redispersed in 1×TBE. The ligand exchange was confirmed by zeta potential measurements.

Functionalization of NR-MHA with single strand DNA (ssDNA). Thiolated ssDNA (SEQ. ID. 1:5′-HS-C6-TTTTTTTTTTTTTTTATAACTTCGTATA-3′ or SEQ. ID. 2:5′-HS-C6-TTTTTTTTTTTTGTCCGGGTCCAGGGC-3′, purchased from Sigma-Aldrich) were deprotected with 100-fold excess of tris(2-carboxyethyl)phosphine (TCEP) over ssDNA. The NR suspension (0.5 mL; 0.5 nM) was incubated with the deprotected ssDNA in a molar ratio of 1:400 in 10 mM phosphate buffer containing 0.3% (w/v) of sodium dodecyl sulfate (SDS) In order to compensate for the repulsion between DNA and gold NR, a charge screening was performed after 3 h of incubation by the addition of 22.5 μl of 0.45 M NaCl every 60 min. This was repeated 4 times and the incubation proceeded overnight. The NR suspension was centrifuged at 9000 g, the supernatant was collected and the pellet was resuspended in 10 mM phosphate buffer with 30 mM NaCl.

Labelling of BSA with a fluorescent dye. A solution of BSA (2 mg/mL, 4.8 nmol, in PBS) was mixed with DyLight 488 (50 μg, 49.4 nmol), DyLight 550 (50 μg, 48.07 nmol) or DyLight 650 (50 μg, 46.9 nmol) and kept under orbital shaking for 2 h. After reaction, the solution was dialysed against PBS in a dialysis cassette (MWCO 10 kDa) for 48 h at 4° C. The final protein concentration and degree of labelling were determined by measuring the absorbance in Nanodrop at 280 nm and at the DyLight absorbance maximum.

Preparation of protein conjugated with ssDNA. Protein-ssDNA conjugates were prepared using N-[γ-maleimidobutyryloxy]sulfosuccinimide ester (sulfo-GMBS, Thermo Scientific) as linker. Briefly, a solution of protein (BSA-DyLight at 7.5 μM or β-galactosidase at 3.5 μM in PBS pH 7.4) was reacted with sulfo-GMBS in a 20-fold molar ratio for 30 min at room temperature. The excess of linker was removed by ultrafiltration with Nanosep 30 kDa (Pall Corporation) and the purified protein (7.5 μM) was reacted with thiolated DNA (22.5 μM) in a final volume of 150 μL of PBS for 2 h at room temperature. DNA strands were complementary to the strands immobilized on the NR surface (SEQ. ID. 3:5′-HS-C6-TATACGAAGTTATAAAAAAAAAA-3′; SEQ. ID. 4:5′-HS-C6-TGCCCTGGACCCGGAC-3′). The conjugate was purified by size exclusion HPLC using a Shimadzu-LC-20AD system with a Superdex 200 5/150 GL column (GE Healthcare). PBS was used as eluent at a flow rate of 0.3 mL/min. Labelling of NR-ssDNA with TRITC. Thiol-PEG-amine 1 kDa (Creative PEGworks, 20 nmol) was reacted with tetramethylrhodamine (TRITC, 20 nmol) in 1 mL of 10 mM carbonate buffer at pH 9.0 for 2 h at room temperature. Then 500 μL of NR-ssDNA (0.5 nM) were incubated overnight with thiol-PEG-TRITC in a molar ratio of 1:1000. The excess of fluorophore was removed by centrifugation at 9000 g.

Immobilization of protein-ssDNA conjugates in Au NRs. For the hybridization of complementary oligonucleotide strands conjugated with a protein, a suspension of NR-ssDNA (0.5 nM) was incubated with DNA-protein conjugates (150 nM) for 1 h at 37° C. and then the temperature was slowly decreased to 25° C. The excess of DNA-protein conjugate was removed by centrifugation. The amount of DyLight-BSA immobilized on the NRs was determined by measuring the fluorescence in the supernatant. The amount of β-galactosidase immobilized was determined by measuring the enzymatic activity in the supernatant. Briefly, 50 μL of supernatant or NR suspension were added to 100 μL of o-nitrophenyl β-d-galactopyranoside (ONPG, 13 mg/mL in 0.1M phosphate buffer pH 7.0) and incubated at 37° C. for 30 min in a Synergy HT microplate reader. The absorbance at 420 nm was measured every 3 min.

Light induced release of proteins from Au NRs. A suspension of DL-BSA-dsDNA-AuNR was placed in a 96 well plate and irradiated with a fiber coupled Roithner laser (continuous wave at 785 nm) with different laser powers (0.8, 1.25, 2 W/cm²). After irradiation, the suspension was immediately centrifuged at 9000 g. The fluorescence of the supernatant was measured in order to determine the amount of protein released.

To test the multiple release system, AuNR conjugated with DL₄₈₈-BSA and DL-₅₅₀-BSA were first irradiated for 2 min at 1.25 W/cm². The supernatant was collected and after resuspending the pellet, the suspension was irradiated for further 3.5 min at 2 W/cm².

A release kinetics was also done using AuNR conjugated with beta-galactosidase. For that, after irradiation the supernatant was collected and its enzymatic activity was measured using ONPG as substrate.

Cytotoxicity of BSA-dsDNA_{51.7}-AuNR. SC-1 mouse fibroblasts were grown in DMEM supplemented with 10% fetal bovine serum (FBS), at 37° C. in a fully humidified air containing 5% CO₂. To assess the cytotoxicity of gold NRs, fibroblasts were seeded on a 96 well plate (4×10³ cells/well), left to adhere for 24 h and then incubated with BSA-dsDNA_{51.7}-AuNR for 4 h. After incubation, the cells were washed with medium to remove non-internalized NR. In some conditions, after incubation with BSA-dsDNA_{51.7}-AuNR, the cells were washed and irradiated with a fiber coupled Roithner laser (785 nm). Each well was placed below the end of the fibre and irradiated with a power density of 1.25 and 2 W/cm² for 2 min. Then cells were left in the incubator for 24 h and the ATP production was measured by a Celltiter-Glo Luminescent Cell Viability Assay (Promega).

Uptake kinetics of BSA-dsDNA_{51.7}-AuNR. SC-1 mouse fibroblasts were plated in a 24 well plate at a density of 5×10⁴ cells/well and left to adhere overnight. The cells were incubated with BSA-dsDNA_{51.7}-AuNR (50 μg/mL) for 1, 2, 4, 6 and 24 h. After incubation, in order to remove non-internalized nanorods, the cells were washed three times with PBS, dissociated with trypsin and counted. Finally, the samples were freeze-dried and the amount of gold was determined by inductive coupled plasma mass spectrometry (ICP-MS).

Light-induced release of proteins in cells. SC-1 mouse fibroblasts were grown in DMEM supplemented with 10% fetal bovine serum (FBS), at 37° C. in 5% CO₂. Cells were incubated with 50 μg/mL of NR conjugated with one or two fluorescent proteins (DL₆₅₀-BSA Tm 51.7° C.; DL₄₈₈BSA Tm 68.9° C.). After 4 h incubation, the medium was replaced and cells were irradiated with fiber coupled Roithner laser (785 nm) with different laser powers (1.25; 2 W/cm²) for 2 min. For the dual release experiments, the time between the application of the first and second stimulus was 5 min.

For the transfection studies with βGal-dsDNA_{51.7}-AuNR-, cells were grown in a 15 well IBIDI slide at an initial density of 3000 cells/well for 24 h. After 4 h incubation with NR-DNAβGal at 50 μg/m L, cells were and irradiated with different laser power densities (0.57; 1.25; 2 W/cm²) for 2 min. After irradiation, the activity of beta-galactosidase was determined with a beta galactosidase Detection Kit (Abcam) following the manufacturer's protocol. The presence of beta-galactosidase was also detected by immunocytochemistry. Cells were seeded in gelatin coated coverslips and left to adhere for 24 h. After incubation with NR-DNAβGal, cells were irradiated, fixed with paraformaldehyde 4% (v/v) for 15 min at room temperature and washed three times with PBS. After blocking (PBS solution with 1% BSA), cells were incubated with a rabbit beta-galactosidase antibody (Invitrogen) for 60 min, washed three times with blocking buffer and incubated with alexa-fluor488 conjugated goat anti-rabbit IgG (dilution 1:1000) for 60 min. The excess of antibody was removed by washing with PBS before staining with DAPI (1 μg/mL) for 5 min. Coverslips were analyzed in a confocal microscope (LSM 710, Carl Zeiss). The corrected total cell fluorescence was quantified with ImajeJ and corrected for background fluorescence. Manders overlap coefficient was calculated using Image J and JACoP plugin.

TABLE 1
Oligonucleotide strands used for conjugation with AuNRs and for
modification of microRNAs. The melting temperature refers to a
theoretical melting temperature relative to the portion of the
strand that is able to hybridize with the complementary strand

| | Tm/° C.* | Sequences |
|---|---|---|
| AuNR conjugation | 51.7 | SEQ. ID 1 |
| | 68.9 | SEQ. ID 2 |
| miR ssDNA conjugates or | 51.7 | SEQ. ID 3 |
| protein-ssDNA conjugates | 68.9 | SEQ. ID 4 |

*melting temperature of complementary strands after hybridization (SEQ. ID3 hybridizes with SEQ. ID1 and SEQ. ID4 hybridizes with SEQ. ID2)

Preparation of Micro-RNAs Conjugated with ssDNA.

microRNAs conjugated with ssDNA (miR-ssDNA) were prepared using N-[γ-maleimidobutyryloxy]sulfosuccinimide ester (sulfo-GM BS, Thermo Scientific) as linker. miR-155 or miR-302a (60 μl at 100 μM in PBS pH 8.0) were reacted with sulfo-GM BS in a 100-fold molar ratio for 30 min at room temperature. The excess of linker was removed by ultrafiltration with Nanosep 30 kDa (Pall Corporation). The buffer was exchanged by PBS pH 7.0 and the purified miR (60 μl; 100 μM in PBS pH 7.0) was reacted with thiolated DNA (60 μl; 200 μM in PBS pH 7.0) in a final volume of 200 μl of PBS for 2 h at room temperature. Before conjugation DNA strands were reduced with 100-fold excess of TCEP for 1 h at 37° C. DNA strands were complementary to the strands immobilized on the NR surface (Table 1) (SEQ. ID. 3:5′-HS-C6-TATACGAAGTTATAAAAAAAAAA; SEQ. ID. 4:5′-HS-C6-TGCCCTGGACCCGGAC). miR-155 was conjugated with ssDNA SEQ. ID. 3(Tm 51.7° C.) and miR-302a was conjugated with ssDNA SEQ. ID. 4 (Tm 68.9° C.).

Purification of miR-ssDNA conjugates by reverse-phase ion-pair liquid chromatography. The products of miR and ssDNA conjugation were separated in a Shimadzu-LC-20AD system using a 4.6×250 mm XBridge C18 column packed with 3.5 μm particles, average pore diameter 130 Å (Waters). The mobile phases were as follows: 0.1 M TEAA pH 7.0 (A) and acetonitrile (B). The gradient started in 14% of B to 19% of B in 23 min. The flow rate was 0.55 mL/min. The acetonitrile present in the fraction containing the miR-ssDNA conjugate was removed in a rotary evaporator. The final volume was aliquoted and stored at −20° C.

Characterization of miR Conjugated with ssDNA by Non-Denaturing PAGE.

The reaction mixture obtained after reacting miR-155 or miR-302 with ssDNA and the fractions obtained after HPLC purification of the reaction mixture were analysed by gel electrophoresis. Reaction mixture (15 μL) and reaction mixture fractions obtained after HPLC purification (15 μL) were mixed with glycerol (5 μl; glycerol in 50% v/v of H₂O), loaded in a polyacrylamide gel (12%, w/v) and run for 45 min in 0.5×TBE at 140 V. The gel was stained with SyBr Gold (1:5000 in 1×TBE) for 10 min and imaged in a UV transilluminator (Molecular Imager Gel DOC, Biorad).

Immobilization of miR-ssDNA Conjugates in AuNRs.

For the hybridization of complementary oligonucleotide strands conjugated with miR, a suspension of AuNR-ssDNA (0.5 nM) was incubated with DNA-miR conjugates (200 or 400 nM) for 1 h at 37° C. and then the temperature was slowly decreased to 25° C. The excess of DNA-protein conjugate was removed by centrifugation. The amount of miR-ssDNA immobilized on the AuNRs was determined indirectly by measuring the concentration in the supernatant. For that, the supernatant was collected and incubated with SyBr Gold (diluted 1:10000). The fluorescence was measured in a Synergy HT microplate reader (excitation 495 nm, emission 537 nm) and the concentration was extrapolated from a calibration curve.

Backfill with Thiol-PEG.

After conjugation with miR-ssDNA conjugates, the surface of AuNRs was backfilled with thiolated PEG (2 kDa). Briefly, a suspension of AuNR-DNA-miR (500 μL, 0.5 nM) was incubated with thiol-PEG at 25 μM corresponding to a ratio of 1:50000 between AuNR and thiol-PEG. The reaction proceeded for 5 h at room temperature under orbital agitation. Then, the suspension was centrifuged (9000 g, 30 min) and resuspended in 10 mM phosphate pH 7.4 with 30 mM NaCl. The suspension was stored at 4° C.

Cell Culture.

HEK-293T transfected with a reporter vector were used for activity assays with microRNAs. The reporter vector encodes EGFP conjugated to the targets of miR-302a and miR-302d, and mCherry conjugated to the targets of miR-142-3p, miR-155 and miR-223. Cells were cultured in T-75 culture flasks at 37° C. in a humidified atmosphere of 5% CO₂ in DMEM cell culture media containing 10% fetal bovine serum (FBS) and 0.5% penicillin-streptomycin. Cells were grown to 80-90% confluency before splitting and re-seeding 24 h before each experiment.

Cytotoxicity of miR-dsDNA-AuNR and Cecropin-Melittin.

To assess the cytotoxicity of AuNRs, HEK-293T cells were seeded on a 96 well plate (12×10³ cells/well), left to adhere for 24 h and then incubated with dsDNA_{51.7}-AuNR (50 μg/mL) without or with cecropin-melttin (5 μM and 10 μM) for 4 h in serum free medium. After incubation, cells were washed with PBS to remove non-internalized AuNRs. In some conditions, after incubation with AuNRs, cells were washed and irradiated with a 780 nm laser at 1.25 W/cm² for 2 min. After 2 h in the incubator at 37° C., a subset of samples received a second laser stimulus for 2 min at 2 W/cm². Then, cells were left in the incubator for 24 h and the ATP production was measured by a Celltiter-Glo Luminescent Cell Viability Assay (Promega) according to the manufacturer's instructions.

Transfection of Micro-RNAs and DNA-miR Conjugates with Lipofectamine RNAimax.

The ability of miR-155, miR-302a and miR-ssDNA conjugates to induce the knockdown of mCherry and EGFP respectively, was evaluated via transfection with lipofectamine RNAimax. HEK-293-T cells were seeded in a collagen coated 96 well plate (6500 cells/well) in DMEM (10% FBS, without antibiotics) 24 h before transfection. miR-155, miR-302a, ssDNA-miR-155 and ssDNA-miR-302a (35 μL, concentrations ranging from 0.05 to 5 nM) were complexed for 20 min with lipofectamine RNAimax diluted in DMEM (0.7 μL of RNAimax in 35 μL of DMEM). Then, each of the complexes was added to cells (20 μL/well) and incubated for 4 h or 24 h. Finally, cells were washed, new culture medium was added and cell fluorescence was monitored in a high-content fluorescence microscope (IN Cell 2200, GE Healthcare) each 12 h during 3 days.

Activity of DNA-miR Conjugates Released from AuNRs.

The activity of miR-155 and miR-302a released from AuNR surface after irradiation was evaluated in HEK-293T cells seeded in a 96 well plate (6500 cells/well). In order to study the laser induced release and activity of miR-DNA conjugates, we used AuNRs hybridized with: I) miR-155 conjugated with ssDNA with a melting temperature of 51.7° C. and II) miR-302a conjugated with ssDNA with a melting temperature of 68.9° C. Each suspension of miR-dsDNA-AuNR was irradiated and immediately centrifuged. Then the supernatant was complexed for 20 min with lipofectamine RNAimax (35 μL of supernatant complexed with 35 μL of RNAimax diluted 1:50 in DMEM) and 20 μL of this mixture was added to each well containing 100 μL of DMEM with 10% FBS. After incubation, cells were kept in DMEM (10% FBS, 0.3 μg/mL of Hoechst) and their fluorescence was monitored in a high-content fluorescence microscope (IN Cell 2200, GE Healthcare) each 12 h during 3 days.

Intracellular Localization of miR-dsDNA-AuNR-TRITC.

Cells were seeded in an IBIDI 15 well slide (80% confluency), left to adhere for 24 h and then incubated with miR-dsDNA-AuNR-TRITC (50 μg/mL) for 4 h without or with cecropin-melittin (5 or 10 μM) in DMEM (0.5% penstrep, without FBS). After incubation, cells were washed with medium to remove non-internalized AuNRs. Then, the cells were incubated with LysoTracker® Green (100 nM) for 30 min to stain the endosomes and with Hoechst 33342 (0.3 μg/m L) to stain the nuclei. Cells were then observed under confocal microscope. Images were analyzed in Image) and the colocalization was determined by calculating the Manders' colocalization coefficient between AuNR-TRITC and Lysotracker green.

Light-Induced Release of DNA-miR Conjugates in Cells.

HEK cells were seeded in a 96 well plate (6500 cells/well), left to adhere for 24 h and then incubated with 50 μg/mL of AuNR conjugated with miR-155-ssDNA or miR-302a-ssDNA. First, AuNRs modified with miR-155 or miR-302a were tested separately. A suspension of miR-dsDNA-AuNR was prepared in serum free DMEM. Before adding to cells, the suspension was mixed with cecropin-melittin peptide (final concentration of 5 or 10 μM). After 4 h incubation, the medium was replaced and cells were irradiated with a fiber coupled laser (780 nm) at 0.8, 1.25 or 2 W/cm² for 2 min. Then, cells were incubated in DMEM (10% FBS, 0.5% penstrep and 0.3 μg/mL of Hoechst 34580) and cell fluorescence was monitored in a high-content fluorescence microscope (IN Cell 2200, GE Healthcare).

All references recited in this document are incorporated herein in their entirety by reference, as if each and every reference had been incorporated by reference individually.

Where ranges are given, endpoints are included. Furthermore, it is to be understood that unless otherwise indicated or otherwise evident from the context and/or the understanding of one of ordinary skill in the art, values that are expressed as ranges can assume any specific value within the stated ranges in different embodiments of the invention, to the tenth of the unit of the lower limit of the range, unless the context clearly dictates otherwise. It is also to be understood that unless otherwise indicated or otherwise evident from the context and/or the understanding of one of ordinary skill in the art, values expressed as ranges can assume any subrange within the given range, wherein the endpoints of the subrange are expressed to the same degree of accuracy as the tenth of the unit of the lower limit of the range.

The disclosure should not be seen in any way restricted to the embodiments described and a person with ordinary skill in the art will foresee many possibilities to modifications thereof.

Where ranges are given, any value within the range may explicitly be excluded from any one or more of the claims. Any embodiment, element, feature, application, or aspect of the compositions and/or methods of the invention, can be excluded from any one or more claims.

The present disclosure should not be seen in any way restricted to the embodiments described and a person with ordinary skill in the art will foresee many possibilities to modifications thereof. The above described embodiments are combinable. The following claims further set out particular embodiments of the disclosure.

1. A composition for controlled release of biomolecules comprising:
a gold nanoparticle;
at least two different biomolecules;
at least two different oligonucleotides having different melting points, binding said biomolecules to the surface of the gold nanoparticle;
wherein the binding is obtainable via hybridization of complementary oligonucleotides;
wherein each of the oligonucleotides is a photo-active such that the respective biomolecule is releasable by photo-activation.

2. The composition according to claim 1 wherein the biomolecule is selected from a list consisting of: a peptide, a protein a micro RNA, a mRNA, and mixtures thereof.

3. The composition according to the previous claim 1 wherein the oligonucleotide sequences comprise between 13-30 base pairs, preferably 13-16 base pairs.

4. The composition according to the previous claims wherein the melting point of a oligonucleotide sequence of the plurality of oligonucleotides sequences varies between 40-90° C., preferably between 50-70° C.

5. The composition according to the previous claims wherein a first oligonucleotide sequence of the plurality of oligonucleotides sequences has a melting point of 50-55° C., and a second oligonucleotide sequence of the plurality of oligonucleotides sequences has a melting point of 65-70° C.

6. The composition according to the previous claims wherein the plurality of oligonucleotides sequences further comprise a poly thymine spacer.

7. The composition according to the previous claims wherein the gold nanoparticle is a nanorod.

8. The composition according to the previous claims comprising at least two oligonucleotides, wherein each oligonucleotide is a sequence 95% identical to: SEQ. ID. 1; SEQ. ID. 2; SEQ. ID. 3; SEQ. ID. 4; and mixtures thereof.

9. The composition according to the previous claims comprising at least two oligonucleotides, wherein each oligonucleotide is a sequence identical to: SEQ. ID 1, SEQ. ID 2, SEQ. ID 3, SEQ. ID 4, or mixtures thereof.

10. The composition according to the previous claims further comprising a cell transfection enhancer.

11. The composition according to the previous claim wherein the cell transfection enhancer is a penetrating peptide, in particular cecropin melitin.

12. The composition according to the previous claims wherein the density of oligonucleotide per gold nanoparticle is between 10-400.

13. The composition according to the previous claims wherein the composition is activated with light wavelengths between 600-1000 nm.

14. The composition according to the previous claims wherein the gold nanoparticle has a size between 10-45 nm, preferably a size between 15-30 nm.

15. The composition according to the previous claims the gold nanoparticle has aspect ratio of 1.5-10, preferably between 3-3.4.

16. The composition according to any one of the previous claims, wherein the composition is a topic formulation or an injectable formulation.

17. The composition according to the previous claims for the use in medicine or veterinary.

18. The composition according to the previous claims for the use in regenerative medicine.

19. The composition according to the previous claims for the use in cell reprograming, in particular adult stem cells, and/or embryonic stem cells.

20. The composition according to the previous claims for the use in the treatment of diseases that respond positively to cell reprograming.

21. The composition according to the previous claims for the use in the treatment of retinal diseases, neurodegenerative diseases, or viral infections.

22. The composition according to the previous claims for the use in the treatment of cancer, aging diseases, and accelerated aging syndromes, infectious diseases such as AIDS, epigenetic diseases such as polycystic ovary syndrome, or cardiac diseases, such as cardiac regeneration.

23. Use of the composition describe in any of the previous claims as a coating of medical devices wherein the composition further comprises an adhesive to immobilize the nanoparticles on top of the medical device.

24. Use of the composition describe in any of the previous claims wherein the medical device is a patch, a catheter, a stent, a contact lens or a pacemaker.


SEQUENCE LISTING (4 sequences)

SEQ ID NO: 1 - DNA, 28 bp, Artificial sequence
  designed sequence with a melting temperature 51.7 Degrees Celsius
tttttttttttttttataacttcgtata

SEQ ID NO: 2 - DNA, 27 bp, Artificial sequence
  designed sequence with a melting temperature 68.9 Degrees Celsius
ttttttttttttgtccgggtccagggc

SEQ ID NO: 3 - DNA, 23 bp, Artificial sequence
  designed sequence with a melting temperature 51.7 Degrees Celsius
tatacgaagttataaaaaaaaaa

SEQ ID NO: 4 - DNA, 16 bp, Artificial Sequence
  designed sequence with a melting temperature 68.9 Degrees Celsius
tgccctggacccggac